(19) United States
(12) Patent Application Publication
(10) Pub. No.: US 2017/0011526 A1
(43) Pub. Date: Jan. 12, 2017

(54) METHOD AND MAGNETIC RESONANCE SYSTEM FOR SEGMENTING A BALLOON-TYPE VOLUME

(71) Applicant: Siemens Healthcare GmbH, Erlangen (DE)

(72) Inventors: Alexander Brost, Erlangen (DE); Christoph Forman, Erlangen (DE); Tanja Kurzendorfer, Trautmannshofen (DE)

(73) Assignee: Siemens Healthcare GmbH, Erlangen (DE)

(21) Appl. No.: 15/203,109

(22) Filed: Jul. 6, 2016

(30) Foreign Application Priority Data
Jul. 6, 2015 (DE) 102015212596.3

(51) Int. Cl. G06T 7/00; A61B 5/055

(52) U.S. Cl. CPC G06T 7/0085; A61B 5/055; G06T 7/0081; G06T 7/0012; G06T 2207/10088; G06T 2207/30048

(57) ABSTRACT

In a method and magnetic resonance apparatus for segmenting a balloon-type volume having an inner surface and an outer surface in an image data record, that is provided to a computer, the image data record at least partially mapping a balloon-type volume, the computer is provided with a starting area and determines a first boundary surface as an inner surface of the balloon-type volume. The computer is provided with a starting surface in the balloon-type volume, and determines a second boundary surface as an outer surface of the balloon-type volume on the basis of the starting surface. The balloon-type volume is determined in the computer as a volume within the first boundary surface and the second boundary surface.

### BACKGROUND OF THE INVENTION

Field of the Invention

The invention concerns a method and a magnetic resonance system (apparatus) for segmenting a balloon-type volume having an inner surface and an outer surface in a three-dimensional image data record.

Description of the Prior Art

With image data records acquired using an imaging modality such as a computed tomography apparatus or a magnetic resonance apparatus, there is frequently a need for automated segmentation, i.e. detection of delimited image areas, the basis of which is formed in most cases by a different image content.

Depending on whether the image is a two-dimensional image or a three-dimensional image, this may involve segmenting surfaces or segmenting volume elements.

A large number of methods are known with which image data records can be automatically segmented. For instance, an image data record can be examined using pattern recognition methods. Segmentation can accordingly be performed by the sought pattern being differentiated from the rest of the image data record, and is thus segmented.

With three-dimensional image data records, difficulties in terms of segmentation increase significantly because translational and rotational as well as deformation movements can take place in all directions.

Balloon-type volumes are volumes that are predominantly curved and at least partially enclose a space. Balloon-type volumes are therefore neither spherical nor necessarily completely closed. They also do not need to be rigid, but can form the basis of a periodic or aperiodic movement. The left ventricle of the heart and the myocardium of the left ventricle of the heart are examples of balloon-type volumes. Whether the myocardium by itself, or together with adjacent tissues such as the epicardium and the endocardium, are among the volumes to be segmented depends on the diagnostic question to be answered, the type of imaging, and other factors. The type of imaging influences the question of the volume to be segmented because different types of imaging map tissue in different ways, and in particular the contrasts between different tissues can be different. If the image data record does not allow a difference to be identified between two adjacent tissues of organs such as the liver and stomach, a segmentation of the liver (for instance) cannot take place. Each volume that at least partially encloses a space thus can be regarded as a balloon-type volume.

There is a particular interest in automatically segmenting the myocardial tissue in magnetic resonance image data records, in order to be able to automatically evaluate scar tissue, which can be displayed highlighted using late gadolinium enhancement (LGE).

### SUMMARY OF THE INVENTION

An object of the present invention is to provide a method of the type noted above wherein an automatic or semiautomatic segmentation of a balloon-type volume is better enabled.

This object is achieved in accordance with the invention by a method for automatically or semi-automatically segmenting a balloon-type volume having an inner surface and an outer surface in an image data record, having the steps:
a) providing an image data record to a computer, which at least partially maps a balloon-type volume,
b) providing a starting area within the balloon-type volume to the computer,
c) determining a first boundary surface in the computer as an inner surface of the balloon-type volume to the computer,
d) providing a starting surface in the balloon-type volume to the computer,
e) determining a second boundary surface in the computer as an outer surface of the balloon-type volume based on the starting surface, and
f) ascertaining the balloon-type volume as a volume within the first boundary surface and the second boundary surface, and making an electronic signal that represent the balloon-type volume available from the computer.

The core of the invention is that the boundary surfaces of the balloon-type volume are defined and the position of the balloon-type volume is thus finally also known. This proceeds from the inside to the outside.

Also applicable are the definition described in the introduction and the cited examples of a balloon-type volume. In addition, the right heart ventricle, the atria of the heart, and the liver are examples of other balloon-type volumes.

The starting area may be a point or a volume. A pixel in a two-dimensional image data record and a voxel in a three-dimensional image data record are such a point. If the starting area is automatically predetermined, this involves an automatic method. If the starting area is alternatively predetermined by a user, this is referred to as a semiautomatic method. The starting area is in any case disposed within the balloon-type volume and therefore not in the sought volume itself. Since the balloon-type volume is predominantly curved, it encloses a volume. The starting area is disposed in this enclosed volume. “Within” is used below to refer to the enclosed volume, “in the balloon-type volume” is the volume occupied by the balloon-type volume itself.

With these definitions, the starting surface is disposed in the balloon-type volume, i.e. it is disposed radially outside of the starting area and also of the first boundary surface.

The balloon-type volume is defined by the definition of the first and second boundary surface. As described, this proceeds from the inside to the outside. In particular, the second boundary surface is disposed radially outside of the balloon-type volume and radially outside of the first boundary surface. In this case the structure needs to be neither spherical nor symmetrical, as already mentioned; radial is only defined by way of the distance from the starting area.

The steps c) to f) are always performed in an evaluation processor. Only step b) may require the help of a user.

An edge detection method can be used to determine the first boundary surface. Such methods isolate planar areas in an image by segmenting them along lines with the aid of different color or gray values. In particular an “active contour” method can be used. These methods are also referred to as “snakes”. Here the object contour is described by parametric curves and corrected by way of energies. Different models and functions can be used for these energies.

An estimated boundary surface can preferably be calculated on the basis of the starting area and the first boundary surface can be calculated on the basis of the estimated boundary surface. In particular, an “active contour” method, as just described, can be used to ascertain the estimated boundary surface. I.e. the “active contour” method is not the very last step in determining the first boundary surface, but instead a step which follows the definition of the starting area. On the basis of the estimated boundary surface, at least one further step then follows for defining the first boundary surface.

The estimated boundary surface or the entire three-dimensional image data record and the estimated boundary surfaces can be converted into polar coordinates for each slice and the calculation of the first boundary surface into polar coordinates can be performed. The use of polar coordinates has also proven to be advantageous in non-cylindrical balloon-type volumes. The center of gravity of the estimated boundary surface can be used as a center of the image in polar coordinates.

The first boundary surface can be ascertained from the estimated boundary surface using a second edge detection method. The first edge detection method then only has the task of “approximately” determining the shape, while the second edge detection method is more sensitive to color differences.

A Canny method can be used here as a second edge detection method. The Canny method is also known as “Canny algorithm” and “Canny edge detector”. By comparison with “active contour” methods, the Canny method is more insensitive to noise, as a result of which it is particularly advantageous in image data records with a low SNR.

Preferably the starting surface can be ascertained as a function of the first boundary surface. If we assume an even thickness of the balloon-type volume, the starting surface is always parallel to the first boundary surface, i.e. the distance from one point of the first boundary surface to a corresponding point of the starting surface is always the same. The distance is typically determined on the basis of a tangent through the point of the first boundary surface. With a three-dimensional image data record, a tangential surface is similarly used as the basis.

The thickness of the balloon-type volume is naturally never constant over the entire surface; a starting surface would otherwise not be required. However, the thickness is often only variable with short distances, so that the starting surface is a very good starting point. A fixed distance from the first boundary surface and from there outwards can preferably be predetermined, as described, in order to ascertain the starting surface.

An edge detection method is preferably used to ascertain the second boundary surface. A Canny method is preferably used here again, as described.

Advantageously the volume elements ascertained with the edge detection method, also known as voxels, can be smoothed by a polynomial regression. For example, the second boundary surface is smoothed. It has namely been shown that the second boundary surface is not as accurately defined by the edge detection method as the first boundary surface. Smoothing is therefore advantageous.

Naturally the first boundary surface can also be smoothed with a polynomial regression. However, the use of a number of edge detection methods is preferred with the first boundary surface.

The starting area can preferably be ascertained from a starting point, by a predetermined radius being used to determine a starting sphere as a starting area. The starting point can be manually predetermined or automatically determined. As noted above, a starting point in a three-dimensional image data record is a starting voxel.

Advantageously, myocardial tissue and/or endocardial tissue and/or epicardial tissue can be used as a balloon-type volume, as a first boundary surface and/or as a second boundary surface respectively. The myocardial tissue is then disposed within the first and the second boundary surface, namely within the endocardial tissue and the epicardial tissue.

The myocardial tissue of a left ventricle of the heart can preferably be determined as a balloon-type volume. The method described above is then a method for segmenting myocardial tissue of a left ventricle of the heart. This is only a preferred application, other myocardial tissue or entirely different tissue can also be segmented, as already described.

In more general terms, the balloon-type volume may be an organ or an organ part, which is mapped in the three-dimensional image data record.

A magnetic resonance image data record is preferably used as the image data record. A magnetic resonance image data record is naturally an image data record that was recorded using a magnetic resonance apparatus.

An image data record that has been recorded following administration of a contrast agent to the examination object, which is mapped in the image data record, is preferably used as an image data record. In particular, an LGE magnetic resonance image data record can be used.

Preferably an image data record that was recorded with parameters, in which the signal from the balloon-type volume is minimized, is used as the image data record. This can take place in addition or alternatively to the administration of a contrast agent. One example of the myocardial tissue may be a magnetic resonance image data record, in which an inversion pulse was used and the recording starts when the signal of the myocardial tissue has its zero crossing. This takes place by suitably selecting the inversion time.

A three-dimensional image data record can be used particularly preferably as an image data record. The method can also be performed on a two-dimensional image data record, wherein there is no synchronization across a number of slices. Ultimately with a three-dimensional image data record, many of the described steps are performed on individual slices, with the slices being considered as two-dimensional image data records. With a three-dimensional image data record, a synchronization across a number of slices can still take place beyond the procedure on a two-dimensional image data record.

The above object underlying the invention is also achieved with a magnetic resonance system having a control computer. The control computer is configured to perform the method as described above.

Further advantageous embodiments of the inventive magnetic resonance apparatus correspond to the described embodiments of the inventive method.

The aforementioned methods can be implemented in the control apparatus as software, or as (hard-wired) hardware.

### BRIEF DESCRIPTION OF THE DRAWINGS

FIG. 1 shows a magnetic resonance system in accordance with the invention.

FIG. 2 shows a flowchart for recording a three-dimensional magnetic resonance data record in accordance with the invention.

FIG. 3 shows a three-dimensional image data record.

FIG. 4 shows a first image for explaining the invention.

FIG. 5 shows a cross-section through a cardiac wall.

FIG. 6 shows a second image for explaining the invention.

FIG. 7 shows a third image for explaining the invention.

FIG. 8 shows a fourth image for explaining the invention.

FIG. 9 shows a fifth image for explaining the invention.

FIG. 10 shows an extract from the fifth image.

FIG. 11 shows a flowchart for segmenting the myocardium of a left ventricle of the heart in accordance with the invention.

### DESCRIPTION OF THE PREFERRED EMBODIMENTS

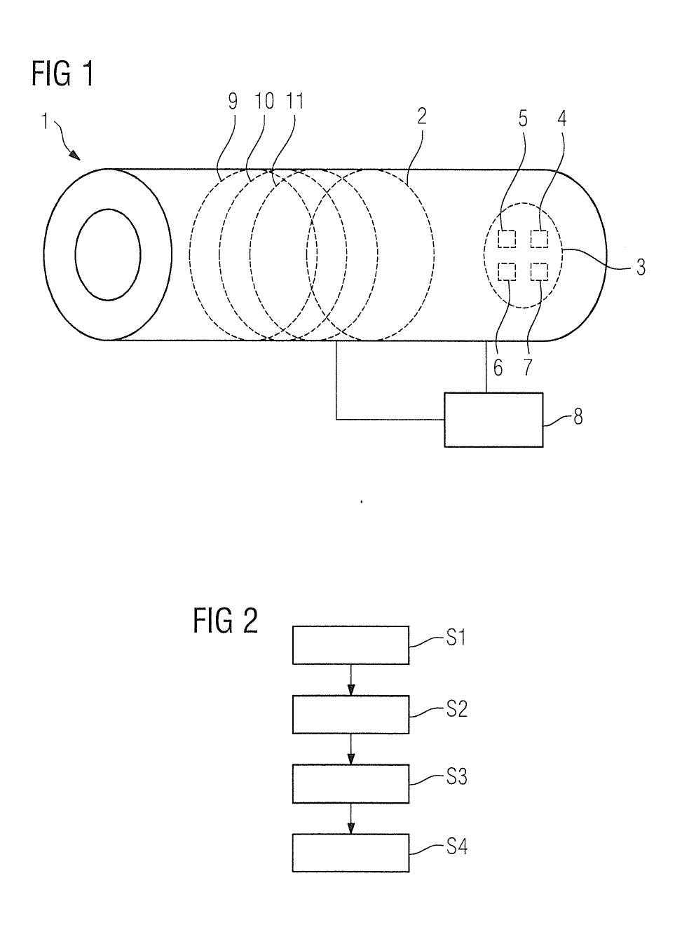

FIG. 1 shows a magnetic resonance apparatus 1. In addition to a radio frequency coil 2 embodied as a body coil, this has a coil array 3 with coils 4, 5, 6 and 7 and a control computer 8. A body coil such as the coil 2 is used to excite the magnetization of nuclear spins. It is therefore also called an excitation coil.

The coil array 3 is used to read out the MR signal that results from the excitation. The coils 4, 5, 6 and 7 of the coil array 3 read out the measured signal simultaneously. This is parallel imaging if more than one coil is used to read out the measured signal. An individual coil can also be used as a detection coil instead of the coil array 3. Particularly with high field devices having a basic magnetic field strength of greater than 10 T and a patient bore of 40 mm to 200 mm, it is common practice to use coils that are simultaneously excitation and detection coils. An image data record can also be recorded herewith, as described further below.

Gradient coils 9, 10 and 11 are required for imaging purposes. The gradient coils 9, 10 and 11 generate gradient fields in three directions. These are overlaid in order to generate the gradients used in a recording sequence, these gradients being in the read, phase and slice direction. Depending on their position, the gradients used in a sequence are composed of the gradients individually or in any combination.

The gradient coils 9, 10 and 11 or the fields generated therewith are, as is known, required for spatial encoding. A phase encoding is produced scanned by repeatedly varying at least one current feed value of one of the gradient coils 9, 10 and 11.

FIG. 2 shows a flowchart for recording a three-dimensional magnetic resonance image data record of a left ventricle of the heart. This can be segmented using the described method for instance.

In step S1 the patient is positioned in the magnetic resonance apparatus 1, wherein the basic settings such as shimming or determining the axis positions are also performed with the aid of scout scans.

A gadolinium-based contrast agent is administered in step S2.

The three-dimensional image data record will be recorded at a predetermined time following administration of the gadolinium in step S3. This can be recorded segmented, i.e. with interruptions. It can however also be acquired “in one step”. A recording method of this type is known for instance from Shin et al., Rapid Single-Breath-Hold 3D Late Gadolinium Enhancement Cardiac MRI Using a Stack-of-Spirals Acquisition, JMRI 40: 1496-1502 (2013). The recording parameters are selected here such that the myocardial tissue provides no signal and thus has minimal or, disregarding the noise signal, no signal values in the image data record. This can be achieved for instance by suitably selecting an inversion time, see above.

Finally, the measured signals thus recorded are processed in step S4; inter alia a Fourier transform takes place so that a three-dimensional image data record 12 is obtained.

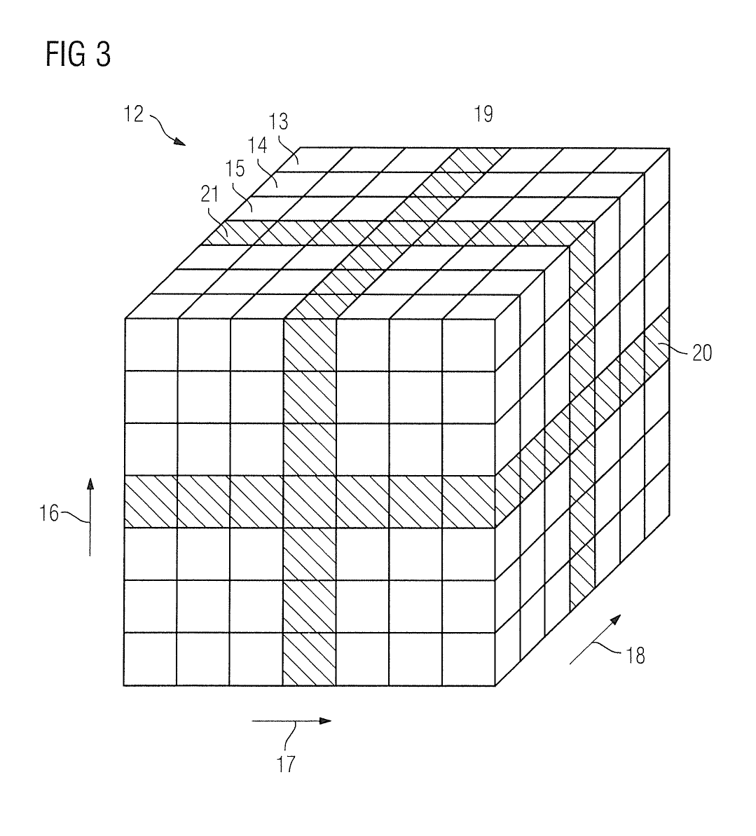

FIG. 3 shows the three-dimensional image data record 12 as a structure made of voxels 13, 14 and 15. The other voxels have no reference numerals. The three directions of the image data record 12 are shown using the arrows 16, 17 and 18. Images in the form of individual slices 19, 20 or 21 can be easily extracted along these directions, which are the read, phase and slice directions. It is known to position the read, phase and slice direction such that the evaluation of the image data record 12 is simplified, for instance by a structure to be examined being intersected vertically by one of the read, phase or slice direction. It can be freely selected as to which of the arrows 16, 17 and 18 shows the read, phase and slice direction, respectively.

Depending on the choice of the gradients and the position of the patient, the 2D images or slices 19, 20 and 21 are sagittal, coronal or axial sectional views or are at an angle hereto.

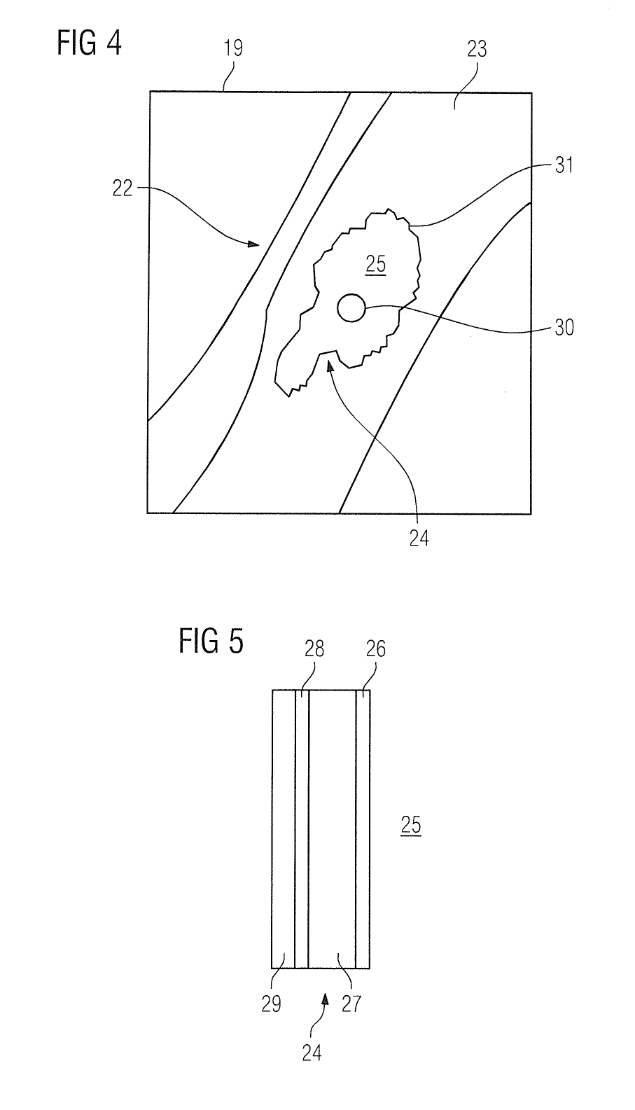

FIG. 4 shows an image of the slice 19 as an example. The left ventricle of the heart 22 and the surrounding tissue 23 are shown schematically. The left ventricle of the heart 22 consists in this sectional view of a strip of tissue 24 within which the space 25 is disposed.

The strip of tissue 24 is illustrated in detail in FIG. 5. The endocardium 26, the myocardium 27 and the epicardium 28 follow from the space 25. The pericardium 29, which for its part is subdivided again, then also appears. However this subdivision has no further relevance to the present application.

The starting area 30 and the estimated boundary surface 31 are also mapped in FIG. 4. On the basis of the starting area 30, the estimated boundary surface 31 is obtained using an “active contour” method for instance.

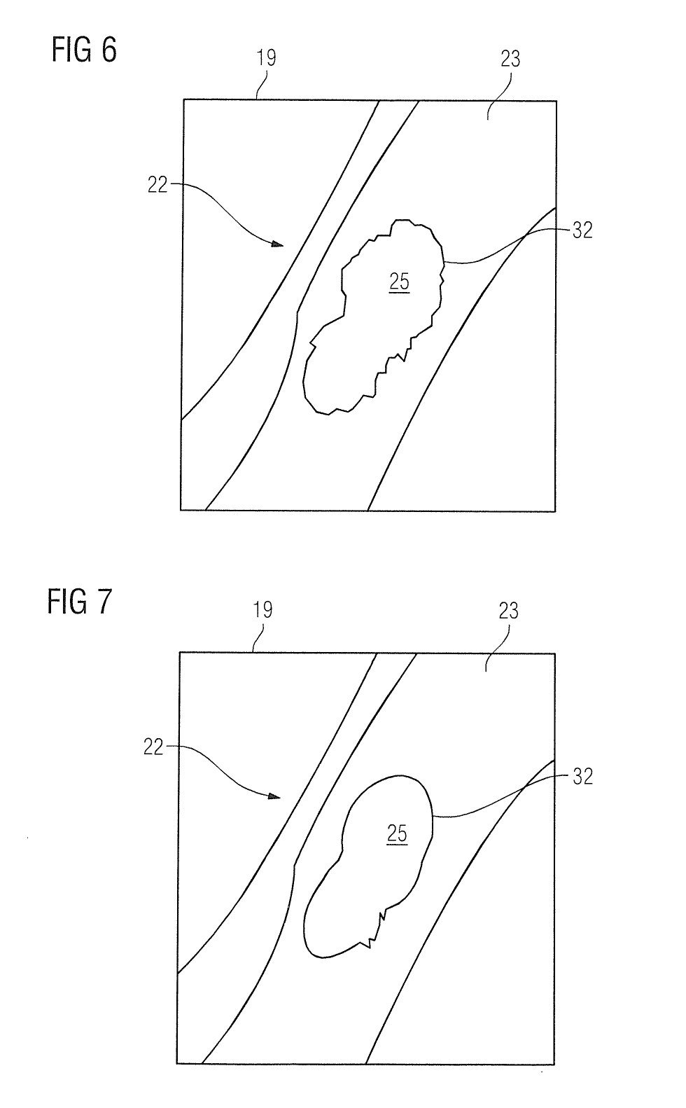

The first boundary surface 32 is ascertained on the basis of the estimated boundary surface 31, by the starting image being transformed into polar coordinates and a further edge detection method such as the Canny method being applied to this polar image. FIG. 6 shows the image 19 with the first boundary surface 32 which is transformed again into Cartesian coordinates. The first boundary surface 32 differs from the estimated boundary surface 31 in the section below to the right for instance, where the notch is omitted.

Prior to the transformation into Cartesian coordinates, the shape of the first boundary surface is preferably smoothed by consideration of the slices parallel to the evaluated slice 19. The first boundary surface 32 which is smoothed in this way is shown in FIG. 7.

Here this first boundary surface 32 is the boundary between the endocardium 26 and the myocardium 27, which is ascertained automatically.

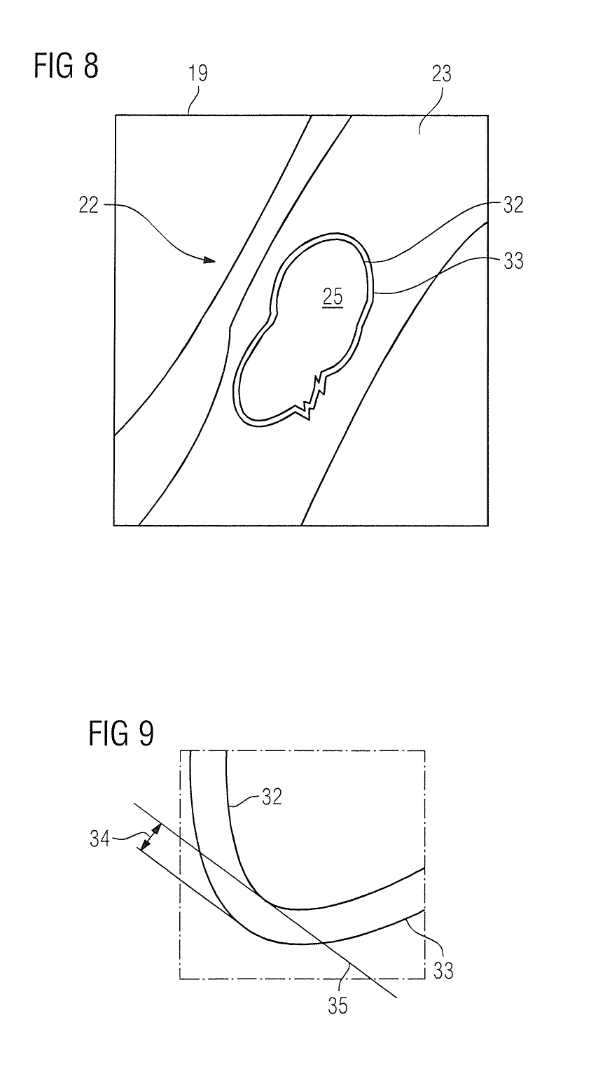

On this basis a starting surface 33 can be ascertained for determining the second boundary surface, by a fixed distance 34 radially outwards being predetermined on the basis of the first boundary surface 32, as FIG. 8 shows. FIG. 9 shows an extract from FIG. 8. The distance 34 is defined at any point on the basis of the tangential surface. Here the tangential surface is a tangent 35 by virtue of the two-dimensional representation.

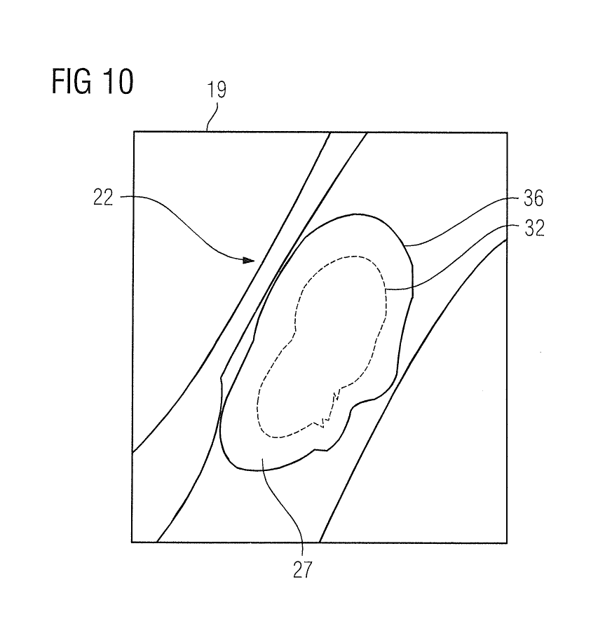

FIG. 10 shows the second boundary surface 36, which was ascertained by means of a Canny method on the basis of the starting surface 33. The first boundary surface 32 is also drawn with dashed lines. The myocardium 27 is then the volume between the first boundary surface 32 and the second boundary surface 36.

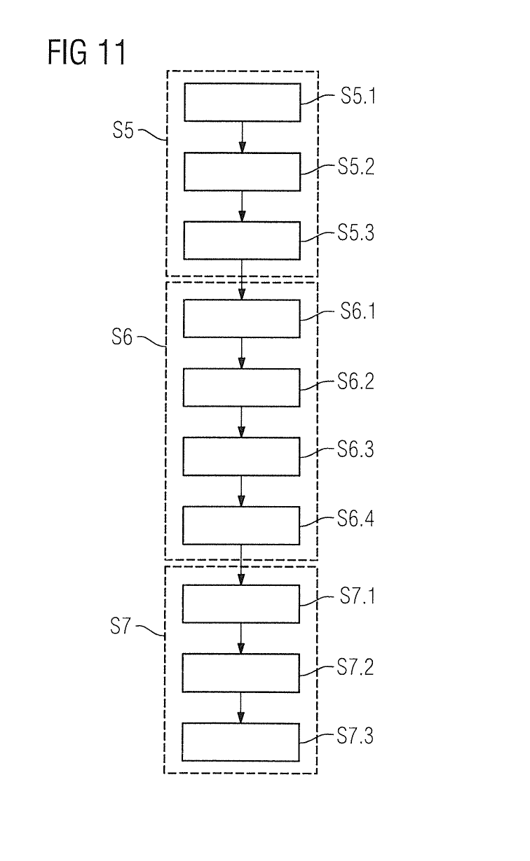

FIG. 11 shows a flowchart for segmenting the myocardium 27 of a left ventricle of the heart 22.

Step S5 with the substeps S5.1 to S5.3 shows the ascertaining of the estimated boundary surface 31.

In step S5.1, a three-dimensional image data record 12 is provided, which maps the left ventricle of the heart 22 and was recorded using an LGE method. In summary, LGE means that a gadolinium contrast agent administration took place before the recording and the signal of the myocardium 27 is suppressed as well as possible by the parameter selection of an inversion module.

Subsequently in step S5.2 a start voxel is predetermined by selecting a voxel in the space 25 within the myocardium 27 and a starting sphere is calculated therefrom as a starting area 30, by a fixed radius being placed around the starting voxel.

On the basis of the starting sphere, the estimated boundary surface 31 is ascertained using an “active contour” method as a first edge detection method in step S5.3.

The determination of the first boundary surface 32 in the form of the endocardium 26 then follows on as step S6 with substeps S6.1 to S6.4.

In step S6.1 a volume around the estimated boundary surface 31 is selected, which is selected generously such that it reliably contains the entire left ventricle of the heart 22 but not the immaterial image areas further outside. This steps helps to save on computing time, but is not compulsory.

In the following step S6.2, the estimated boundary surface 31 and the further voxels of the selected volume are converted into polar coordinates. The center of gravity of the estimated boundary surface 31 is preferably used as a center of the image in polar coordinates.

In step S6.3, the first boundary surface 32 is ascertained by means of a Canny method as a second edge detection method from the estimated boundary surface 31. A factor σ=2 can preferably be used here so that smaller corners are extracted.

The first boundary surfaces 32 thus obtained are smoothed as step S6.4 in the direction vertical to the direction of the slice 19, as for instance in the direction of the arrow 17, by considering the first boundary surfaces 32 found in the adjacent slice or slices. Individual inaccuracies in a slice can be compensated in this way.

In step S7, the epicardial structure is determined.

In step S7.1, a starting surface 33 is predetermined on the basis of the first boundary surface 32, by a fixed distance being plotted radially outwards. The radius is selected such that the starting surface is reliably disposed in the myocardium 27.

In step S7.2, a Canny method is used again in order to define the second boundary surface 36. This is the third time that an edge detection method is used.

The voxels of the second boundary surface 36 thus found are smoothed in step S7.3 by a polynomial regression. The second boundary surface 36 is the boundary surface between the myocardium 27 and the epicardium 28.

The steps S7.1 and S7.2 are also preferably performed in polar coordinates. A conversion into Cartesian coordinates is only appropriate for displaying individual images.

With the described method, the myocardium can be segmented without a further image data record being required. The myocardium has hitherto typically been segmented in another image data record, for instance a Cine data record, and this structure placed in an LGE image data record. It is possible with the described method to segment the myocardium directly in a two-dimensional or three-dimensional LGE image data record.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for segmenting a balloon-type volume having an inner surface and an outer surface in an image data record, comprising:
a) providing an image data record to a computer, said image data record at least partially mapping a balloon-type volume;
b) providing a designation of a starting area to said computer;
c) determining a first boundary surface in said computer as an inner surface of the balloon-type volume;
d) providing a designation of a starting surface in the balloon-type volume to said computer;
e) determining a second boundary surface in said computer as an outer surface of the balloon-type volume based on the starting surface; and
f) ascertaining the balloon-type volume as a volume within the first boundary surface and the second boundary surface, and making an electronic signal that represents the balloon-type volume available from the computer.

2. The method as claimed in claim 1, comprising using an edge detection method in said computer to determine the first boundary surface.

3. The method as claimed in claim 2 comprising using an active contour method as said edge detection method to determine said first boundary surface.

4. The method as claimed in 2, comprising calculating an estimated boundary surface in said computer based on the starting area, and calculating the first boundary surface, in said computer based on, the estimated boundary surface.

5. The method as claimed in claim 4, comprising converting the estimated boundary surface into polar coordinates and calculates the first boundary surface in polar coordinates.

6. The method as claimed in claim 4, comprising determining the first boundary surface from the estimated boundary surface using further edge detection method.

7. The method as claimed in claim 6, comprising using a Canny method as the function edge detection method.

8. The method as claimed in claim 1, comprising determining the starting surface dependent on the first boundary surface.
characterized in that the starting surface (33) is ascertained as a function of the first boundary surface (32).

9. The method as claimed in claim 8, comprising predetermining a fixed distance in order to determine the starting surface.

10. The method as claimed in claim 1, comprising using an edge detection method to determine the second boundary surface.

11. The method as claimed in claim 10, comprising smoothing volume elements ascertained as the second boundary surface with the edge detection method, using a polynomial regression.

12. The method as claimed in claim 1, comprising determining the starting area from a starting point by using a predetermined radius to ascertain a starting sphere as the starting area.

13. The method as claimed in claim 1, determining myocardial tissue, endocardial tissue, and epicardial tissue as the balloon-type volume, the first boundary surface, and the second boundary surface, respectively.

14. The method as claimed in claim 1, comprising determining myocardial tissue of a left ventricle of the heart as the balloon-type volume.

15. The method as claimed in claim 1, comprising using, as said image data record, an image data record that was recorded following administration of a contrast agent to the examination object that is mapped in the image data record.

16. The method as claimed in claim 1, comprising using a three-dimensional image data record as said image data record.

17. A magnetic resonance apparatus comprising:
a magnetic resonance data acquisition scanner;
a control computer configured to operate the magnetic resonance data acquisition scanner to obtain raw data from an examination subject situated in the magnetic resonance data acquisition scanner, said raw data representing a balloon-type volume in the examination subject having an inner surface and an outer surface;
said control computer being configured to reconstruct an image data record from said raw data, said image data record representing said balloon-type volume with said inner surface and said outer surface;
said control computer being configured to receive a designation of a starting area and a starting surface in the balloon-type volume;
said control computer being configured to determine a first boundary surface as said inner surface of said balloon-type volume from said starting area and to determine a second boundary surface, as said outer surface, based on said starting surface; and
said control computer being configured to ascertain the balloon-type volume as a volume within the first boundary surface and the second boundary surface, and to make an electronic signal that represents the balloon-type volume available from the control computer.
